(19) United States
(12) Patent Application Publication
(10) Pub. No.: US 2014/0200451 A1
(43) Pub. Date: Jul. 17, 2014

(54) ULTRASONIC MEASURING DEVICE, ULTRASONIC DIAGNOSIS DEVICE, ULTRASONIC MEASUREMENT SHEET, AND MEASURING METHOD

(71) Applicant: Seiko Epson Corporation, Tokyo (JP)

(72) Inventors: Masaki HAYASHI, Shiojiri (JP); Ryoki WATANABE, Suwa (JP)

(73) Assignee: Seiko Epson Corporation, Tokyo (JP)

(21) Appl. No.: 14/150,978

(22) Filed: Jan. 9, 2014

(30) Foreign Application Priority Data
Jan. 15, 2013 (JP) 2013-004302

(51) Int. Cl. A61B 8/00

(52) U.S. Cl. CPC A61B 8/54; A61B 8/44
USPC 600/437

(57) ABSTRACT

An appropriate parameter can be automatically set according to the subject in an ultrasonic measuring device. The ultrasonic measuring device includes an emission unit that performs ultrasound emission processing, a reception unit that performs ultrasonic echo reception processing, and a processing unit that performs ultrasonic measurement control processing. The emission unit performs processing for emitting ultrasound toward a subject via an ultrasonic measurement sheet, and the reception unit performs processing for receiving ultrasonic echoes from the ultrasonic measurement sheet, and outputs a reception signal to the processing unit. The processing unit performs processing for analyzing ultrasonic measurement code information recorded in the ultrasonic measurement sheet based on the reception signal from the reception unit.

### BACKGROUND

1. Technical Field

The present invention relates to an ultrasonic measuring device, an ultrasonic diagnosis device, an ultrasonic measurement sheet, a measuring method, and the like.

2. Related Art

With ultrasonic diagnosis devices, emission and reception control parameters, image generation parameters, and the like need to be set appropriately in order to obtain ultrasonic images that are appropriate for various diagnosis sites. However, many parameters need to be set, and it is difficult to know how to adjust the parameters in order to obtain images appropriate for diagnosis, thus making it difficult for a user without expertise to appropriately set such parameters.

In light of this issue, JP-A-2001-161695 discloses a technique in which the user selects a body mark that corresponds to the diagnosis site, and the diagnosis apparatus sets image parameters that correspond to the selected body mark.

JP-A-2001-161695 is an example of related art.

### SUMMARY

However, the technique disclosed in JPA-2001-161695 has a problem in that since the user needs to select a body mark himself/herself, there is the risk of inappropriate parameters being set due to a selection error. According to several aspects of the invention, it is possible to provide an ultrasonic measuring device, an ultrasonic diagnosis device, an ultrasonic measurement sheet, a measuring method, and the like that enable appropriate parameters to be automatically set according to the subject.

A first aspect of the invention relates to an ultrasonic measuring device including: an emission unit that performs ultrasound emission processing; a reception unit that performs ultrasonic echo reception processing; and a processing unit that performs ultrasonic measurement control processing, wherein the emission unit performs processing for emitting ultrasound toward a subject via an ultrasonic measurement sheet, the reception unit performs processing for receiving an ultrasonic echo from the ultrasonic measurement sheet, and outputs a reception signal to the processing unit, and the processing unit performs processing for analyzing ultrasonic measurement code information recorded in the ultrasonic measurement sheet based on the reception signal from the reception unit.

According to the first aspect of the invention, the processing unit can perform processing for analyzing ultrasonic measurement code information recorded by the ultrasonic measurement sheet, and therefore it is possible to automatically set parameters for ultrasonic measurement based on the code information. As a result, there is no need for the user to perform a parameter setting operation, and it is possible to set parameters that are appropriate for the subject.

Also, in the first aspect of the invention, the processing unit may set a control parameter that is used in control of at least one of the emission unit and the reception unit based on the code information, and control at least one of the emission unit and the reception unit based on the control parameter.

According to this configuration, the processing unit can control at least one of the emission unit and the reception unit using a control parameter that was set based on the code information, and therefore it is possible to perform emission and reception processing using a control parameter that is appropriate for the subject, for example.

Also, in the first aspect of the invention, the processing unit may set an image generation parameter for generation of ultrasonic image data based on the code information, and generate the ultrasonic image data based on the image generation parameter.

According to this configuration, the processing unit can generate ultrasonic image data using an image generation parameter that was set based on the code information, and therefore it is possible to generate ultrasonic image data using an image generation parameter that is appropriate for the subject, for example.

Also, in the first aspect of the invention, the image generation parameter may be a parameter for setting at least one of gain and dynamic range.

According to this configuration, the processing unit can generate ultrasonic image data that is appropriate for the subject using at least one of a gain and a dynamic range set based on the code information.

Also, in the first aspect of the invention, in a first period, the processing unit may perform the code information analysis processing and, based on the code information, set a control parameter that is used in control of at least one of the emission unit and the reception unit and an image generation parameter for generation of ultrasonic image data, and in a second period that is after the first period, the processing unit may control at least one of the emission unit and the reception unit based on the control parameter, and generate the ultrasonic image data based on the image generation parameter.

According to this configuration, in the first period, the processing unit can set a control parameter and an image generation parameter based on the code information, and in the second period, the processing unit can perform ultrasonic measurement processing that is appropriate for the subject using the control parameter and the image generation parameter that were set.

Also, in the first aspect of the invention, the first period may be a period in which the processing unit sets a parameter to be used in ultrasonic measurement, and the second period may be a period in which the processing unit performs ultrasonic measurement control processing based on the parameter that was set.

According to this configuration, in the first period, the processing unit can set a parameter that is used in ultrasonic measurement, and in the subsequent second period, the processing unit can perform ultrasonic measurement control processing using the parameter that was set.

Also, in the first aspect of the invention, the code information may be site specification information for specifying a measurement site.

According to this configuration, the processing unit can specify the measurement site based on the code information.

Also, in the first aspect of the invention, based on the site specification information, the processing unit may set a control parameter that is used in control of at least one of the emission unit and the reception unit and an image generation parameter for generation of ultrasonic image data, the control parameter and the image generation parameter being parameters that are used in measurement of the measurement site.

According to this configuration, the processing unit can set a control parameter, an image generation parameter, and the like that are appropriate for the measurement site.

Also, in the first aspect of the invention, the processing unit may acquire at least one of characteristic information and manufacturing information of the ultrasonic measurement sheet based on the code information.

According to this configuration, the processing unit can acquire information indicating the thickness, the acoustic impedance, the manufacturing date, the manufacturing number, and the like of the ultrasonic measurement sheet, for example, and therefore it is possible to prevent the user from mistakenly using an inappropriate sheet or a sheet that has deteriorated due to aging.

Also, in the first aspect of the invention, the ultrasonic measurement sheet may have an ultrasound transmissive medium and a plurality of reflectors embedded in the ultrasound transmissive medium, the code information may be recorded using at least one of the reflectance, the number, the shape, and the size of the plurality of reflectors, the reception unit may perform processing for receiving an ultrasonic echo from the plurality of reflectors, and output a reception signal to the processing unit, and the processing unit may perform processing for analyzing the code information based on the reception signal from the reception unit.

According to this configuration, the processing unit can analyze code information that was recorded using at least one of the reflectance, the number, the shape, and the size of the reflectors, and perform appropriate ultrasonic measurement processing.

Also, in the first aspect of the invention, the ultrasonic measurement sheet may have a plurality of reflector groups aligned in the ultrasound transmissive medium as the plurality of reflectors, each reflector group among the plurality of reflector groups may have 1st to p-th (p being an integer greater than or equal to 2) reflectors that are aligned along a depth direction of the ultrasonic measurement sheet, and the processing unit may perform processing for analyzing the code information recorded by the 1st to p-th reflectors.

According to this configuration, the processing unit can analyze code information that was recorded using the 1st to p-th reflectors of a reflector group, and perform appropriate ultrasonic measurement processing.

Also, in the first aspect of the invention, the same code information may be recorded by each reflector group among the plurality of reflector groups, and the processing unit may perform processing for analyzing the code information recorded by at least one reflector group among the plurality of reflector groups.

According to this configuration, the processing unit can acquire the same code information from any reflector group among the reflector groups that the ultrasonic measurement sheet has.

Also, a second aspect of the invention relates to an ultrasonic diagnosis device including: any of the above-described ultrasonic measuring devices; and a display unit that displays display image data.

Also, a third aspect of the invention relates to an ultrasonic measurement sheet including: an ultrasound transmissive medium; and a plurality of reflectors embedded in the ultrasound transmissive medium, wherein code information that is to be analyzed in analysis processing performed by an ultrasonic measuring device is recorded using at least one of the reflectance, the number, the shape, and the size of the plurality of reflectors.

According to the third aspect of the invention, the ultrasonic measurement sheet can record code information, which is information that is to be analyzed, using at least one of the reflectance, the number, the shape, and the size of the reflectors.

Also, in the third aspect of the invention, the ultrasonic measurement sheet may include a plurality of reflector groups aligned in the ultrasound transmissive medium as the plurality of reflectors, each reflector group among the plurality of reflector groups may have 1st to p-th (p being an integer greater than or equal to 2) reflectors that are aligned along a depth direction of the ultrasonic measurement sheet, and the code information may be recorded by the 1st to p-th reflectors.

According to this configuration, the ultrasonic measurement sheet can record code information using each of the reflector groups.

### BRIEF DESCRIPTION OF THE DRAWINGS

FIG. 1 shows an example of basic configurations of an ultrasonic measuring device and an ultrasonic diagnosis device.

FIGS. 2A and 2B show an example of a basic configuration of an ultrasonic measurement sheet.

FIGS. 3A and 3B show examples of methods of manufacturing the ultrasonic measurement sheet.

FIGS. 4A to 4C show examples of specific configurations of the ultrasonic measurement sheet.

FIG. 5A shows an example of use of the ultrasonic measurement sheet. FIG. 5B shows an example of an ultrasonic image (B mode image).

FIG. 6A shows an example of code information recorded using the reflectances of reflectors. FIG. 6B shows an example of a luminance table. FIG. 6C shows an example of a reference table.

FIG. 7 shows an example of an image of a reflector group.

FIGS. 8A and 8B are diagrams for describing gain.

FIG. 9 is a diagram for describing dynamic range.

FIG. 10 is an example of a flowchart for measuring processing performed by the ultrasonic measuring device.

FIG. 11 is an example of a flowchart for code information analysis processing performed by the ultrasonic measuring device.

FIGS. 12A and 12B show examples of specific configurations of an ultrasonic diagnosis device. FIG. 12C shows an example of a specific configuration of an ultrasonic probe.

### DESCRIPTION OF EXEMPLARY EMBODIMENTS

The following is a detailed description of preferred embodiments of the invention. Note that the embodiments described below are not intended to unduly limit the content of the invention recited in the claims, and all of the configurations described in the embodiments are not necessarily essential as solutions provided by the invention.

1. Ultrasonic Measuring Device

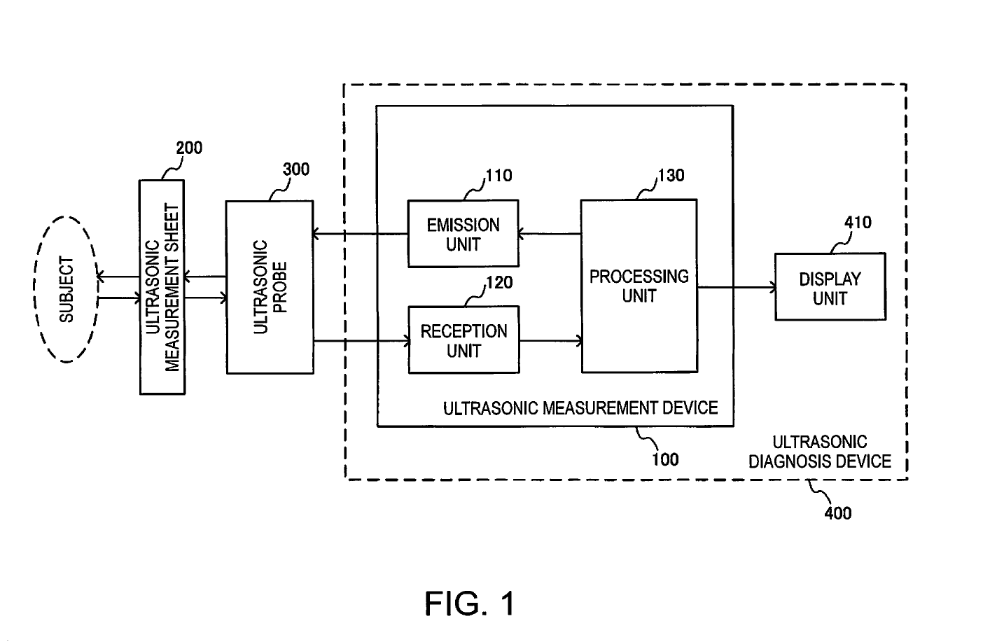

FIG. 1 shows an example of the basic configurations of an ultrasonic measuring device 100 and an ultrasonic diagnosis device 400 of this embodiment. The ultrasonic measuring device 100 of this embodiment includes an emission unit 110, a reception unit 120, and a processing unit 130. Also, the ultrasonic diagnosis device 400 of this embodiment includes the ultrasonic measuring device 100 and a display unit 410. Note that the ultrasonic measuring device 100 and the ultrasonic diagnosis device 400 of this embodiment are not limited to the configurations shown in FIG. 1, and various modifications can be carried out, such as omitting some of the constituent elements, replacing some of the constituent elements with other constituent elements, and adding other constituent elements.

The emission unit 110 performs ultrasound emission processing. Specifically, the emission unit 110 outputs an emission signal (drive signal), which is an electrical signal, to an ultrasonic probe 300, and the ultrasonic probe 300 emits ultrasound toward a subject via an ultrasonic measurement sheet 200. The ultrasonic probe 300 includes an ultrasonic transducer device (not shown), and the ultrasonic transducer device converts the emission signal, which is an electrical signal, into ultrasonic waves.

The reception unit 120 performs ultrasonic echo reception processing. Specifically, the ultrasonic transducer device included in the ultrasonic probe 300 converts ultrasonic echoes from the subject and the ultrasonic measurement sheet 200 into an electrical signal. The reception unit 120 performs reception processing such as amplification, wave detection, A/D conversion, and phase matching on a reception signal (analog signal), which is an electrical signal, from the ultrasonic transducer device, and outputs the reception signal (digital data) that is the result of the reception processing to the processing unit 130.

The processing unit 130 performs ultrasonic measurement control processing. Specifically, the processing unit 130 performs processing for controlling the emission unit 110 and the reception unit 120, and processing for generating ultrasonic image data based on a reception signal from the reception unit 120. The processing unit 130 also performs processing for analyzing ultrasonic measurement code information, which is recorded in the ultrasonic measurement sheet 200, based on the reception signal from the reception unit 120.

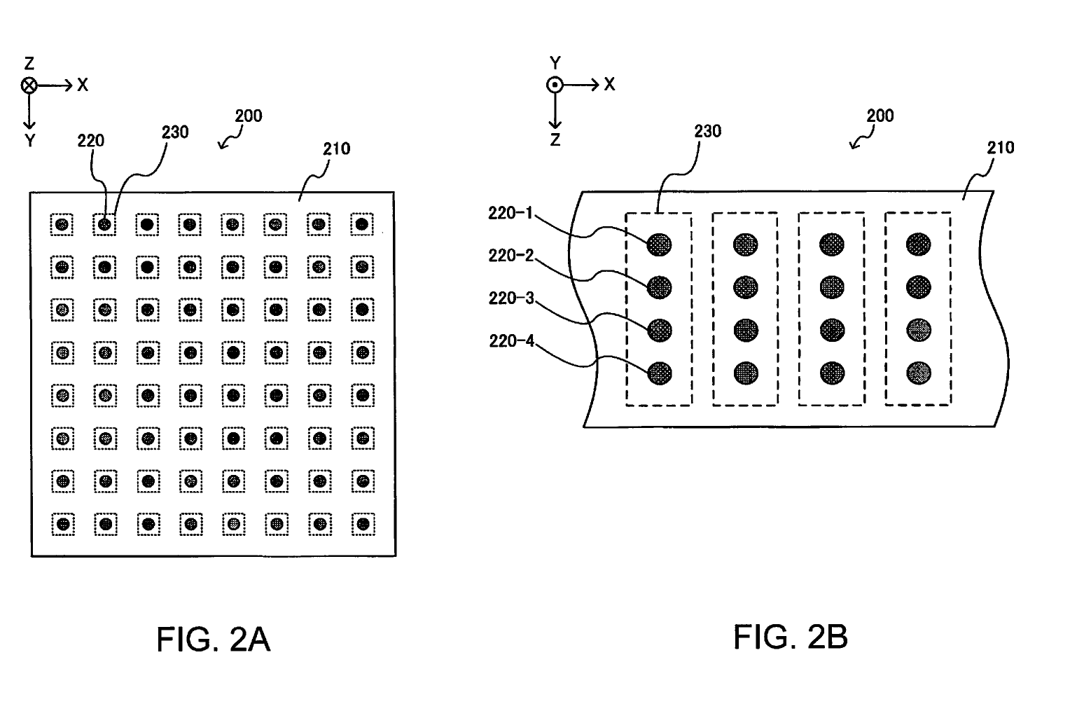

Ultrasonic measurement code information is recorded in the ultrasonic measurement sheet 200 using multiple reflectors (see FIGS. 2A and 2B) that the ultrasonic measurement sheet 200 has. Portions of the ultrasonic waves emitted from the ultrasonic probe 300 are reflected by the reflectors that the ultrasonic measurement sheet 200 has. The processing unit 130 can perform processing for analyzing the code information based on a reception signal that is based on ultrasonic echoes from the reflectors. Details of the code information recorded by the reflectors and details of the code information analysis processing performed by the processing unit 130 will be described later.

The processing unit 130 can set a control parameter that is used in the control of at least one of the emission unit 110 and the reception unit 120 based on the code information, and control at least one of the emission unit 110 and the reception unit 120 based on the control parameter. One example of this control parameter is the frequency of the ultrasonic waves that are to be emitted and received. According to this configuration, a control parameter can be set automatically, and therefore it is possible for the ultrasonic frequency to be set to an appropriate value without the user performing an operation.

Also, the processing unit 130 can set an image generation parameter for the generation of ultrasonic image data based on the code information, and generate ultrasonic image data based on the image generation parameter. This image generation parameter is a parameter for setting at least one of the gain and the dynamic range. According to this configuration, an image generation parameter can be set automatically, and therefore it is possible for the gain, the dynamic range, and the like to be set to an appropriate value without the user performing an operation.

The ultrasonic measurement code information recorded in the ultrasonic measurement sheet 200 is site specification information for specifying the measurement site. According to this configuration, a control parameter and an image generation parameter can be automatically set in accordance with the measurement site, and therefore it is possible for these parameters to be set to values that are appropriate for the measurement site without the user performing an operation.

Also, the processing unit 130 can acquire at least one of characteristic information and manufacturing information of the ultrasonic measurement sheet 200 based on the code information. Characteristic information indicates the thickness, the acoustic impedance, and the like of the ultrasonic measurement sheet 200, for example. Manufacturing information indicates the manufacturing date, the manufacturing number, and the like of the ultrasonic measurement sheet 200, for example. This configuration enables preventing the user from mistakenly using an inappropriate sheet or a sheet that has deteriorated due to aging.

The display unit 410 is a liquid crystal display, for example, and displays display image data from the processing unit 130. This display image data includes ultrasonic image data for a subject.

In this way, according to the ultrasonic measuring device 100 of this embodiment, the processing unit 130 can perform processing for analyzing ultrasonic measurement code information that is recorded in the ultrasonic measurement sheet 200. The processing unit 130 can then set a control parameter that is used in the control of at least one of the emission unit 110 and the reception unit 120 based on the analyzed code information, and control at least one of the emission unit 110 and the reception unit 120 based on the control parameter. Also, the processing unit 130 can set an image generation parameter for the generation of ultrasonic image data based on the analyzed code information, and generate ultrasonic image data based on the image generation parameter. This code information can be site specification information for specifying the measurement site.

Since a control parameter and an image generation parameter can be automatically set in accordance with the measurement site in this way, it is possible for these parameters to be set to values that are appropriate for the measurement site without the user performing an operation.

2. Ultrasonic Measurement Sheet

FIGS. 2A and 2B show an example of the basic configuration of the ultrasonic measurement sheet 200 of this embodiment. The ultrasonic measurement sheet 200 of this embodiment includes an ultrasound transmissive medium 210 and multiple reflectors 220 (220-1 to 220-4). Note that the ultrasonic measurement sheet 200 of this embodiment is not limited to the configuration shown in FIGS. 2A and 2B, and various modifications can be carried out, such as omitting some of the constituent elements, replacing some of the constituent elements with other constituent elements, and adding other constituent elements.

FIG. 2A is a top view of the ultrasonic measurement sheet 200, and FIG. 2B is a cross-sectional view of the ultrasonic measurement sheet 200. As shown in FIGS. 2A and 2B, the direction parallel to one of the sides of the ultrasonic measurement sheet 200 is the X direction, and the direction that is perpendicular to the X direction and parallel to the sheet surface is the Y direction. Also, the direction that is perpendicular to the sheet surface, that is to say the thickness direction of the sheet, is the Z direction.

It is desirable that the ultrasound transmissive medium 210 is formed from a material that transmits ultrasound, has an acoustic impedance close to that of the human body, and has little attenuation. For example, it can be formed from an oil gel, acrylamide, a hydro gel, or the like. This ultrasound transmissive medium 210 is used in close contact with the human body (test subject).

The reflectors 220 are formed from a material that has a different acoustic impedance from the ultrasound transmissive medium 210, and they are embedded in the ultrasound transmissive medium 210. Due to having a different acoustic impedance from the ultrasound transmissive medium 210, the reflectors 220 reflect ultrasonic waves. Rubber or the like can be used as the material for the reflectors 220. The code information is recorded using at least one of the reflectance, the number, the shape, and the size of the reflectors 220. Specifically, the code information is recorded by setting at least one of the reflectance, the number, the shape, and the size to a predetermined value. For example, the code information can be recorded by setting the reflectances of the reflectors 220 to any of multiple predetermined reflectances.

Letting Z1 be the acoustic impedance of the ultrasound transmissive medium 210, and Z2 be the acoustic impedance of a reflector 220, the reflectance R of that reflector 220 is obtained with the following equation.

R=(Z2−Z1)/(Z1+Z2) (1)

Also, the acoustic impedance Z is obtained with the following equation.

Z=ρ×c (2)

Here, ρ is the density of the medium, and c is the acoustic velocity in the medium.

Accordingly, the acoustic impedance Z2 can be set variably by changing the material used for the reflectors 220. For example, it is possible to use silicone-based rubber or the like as the base material for the reflector 220, and change the acoustic impedance Z2 of the reflector 220 by mixing in a filler such as a metal. Specifically, the reflectance R of the reflector 220 can be set to any of four levels by changing the proportion of the filler between four levels. The greater the amount of the filler is, the closer the acoustic impedance approaches the acoustic impedance of the filler.

The ultrasonic measurement sheet 200 may include multiple reflector groups 230 that are aligned in the ultrasound transmissive medium 210 as the reflectors 220. Each of the reflector groups 230 includes 1st to p-th (p being an integer greater than or equal to 2) reflectors that are aligned along the depth direction (Z direction) of the ultrasonic measurement sheet 200. The reflector group 230 shown in FIG. 2B includes first to fourth reflectors 220-1 to 220-4, for example. Each reflector group 230 can record code information using the first to fourth reflectors 220-1 to 220-4 included therein. The processing unit 130 performs processing for analyzing the code information recorded using the first to fourth (in a broad sense, p-th) reflectors 220-1 to 220-4.

The same code information may be recorded by each of the reflector groups 230. For example, the same code information may be recorded by all of the reflector groups 230 shown in FIG. 2A. The processing unit 130 performs processing for analyzing the code information recorded by at least one reflector group among the reflector groups 230. According to this configuration, the processing unit 130 can acquire the same code information regardless of which portion of the ultrasonic measurement sheet 200 the ultrasonic probe 300 comes into contact with.

Although FIG. 2A shows an example in which the reflector groups 230 are arranged in a matrix, the arrangement is not limited to this. For example, a staggered arrangement may be used, or the reflector groups 230 may be arranged in concentric circles. Although FIG. 2B shows an example in which each reflector group 230 has first to fourth reflectors, the number of reflectors 220 that make up one reflector group 230 is not limited to this.

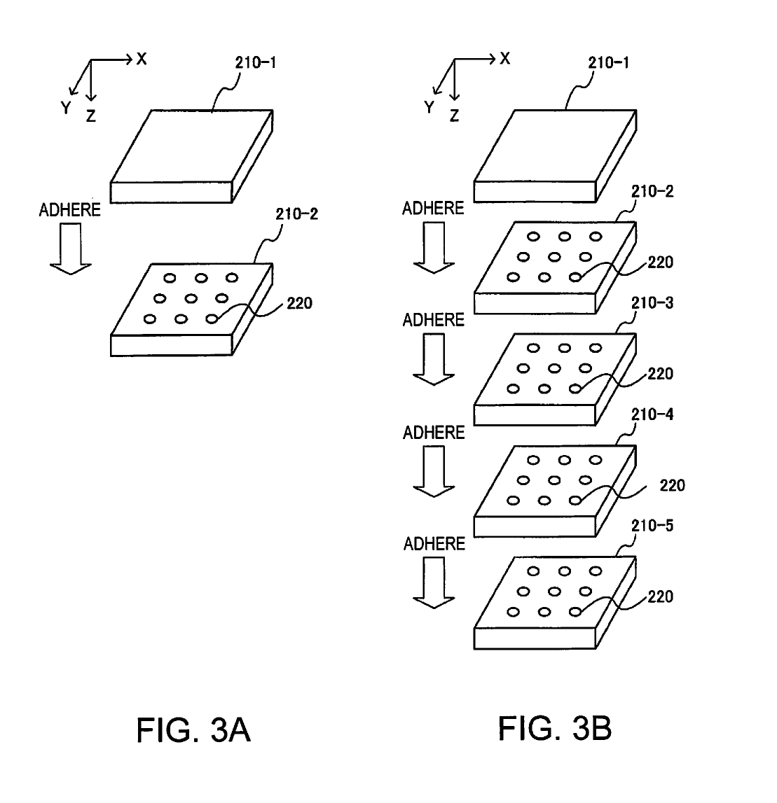

FIGS. 3A and 3B show examples of methods of manufacturing the ultrasonic measurement sheet 200 of this embodiment. As shown in FIG. 3A, the ultrasonic measurement sheet 200 having multiple reflectors 220 can be manufactured by adhering an ultrasound transmissive medium 210-1 that does not have reflectors 220 onto an ultrasound transmissive medium 210-2 on which multiple reflectors 220 are arranged.

FIG. 3B shows an example of a method of manufacturing an ultrasonic measurement sheet 200 that has multiple reflector groups 230. An ultrasound transmissive medium 210-4 on which multiple reflectors 220 are arranged is adhered onto an ultrasound transmissive medium 210-5 on which multiple reflectors 220 are arranged, and then an ultrasound transmissive medium 210-3 on which multiple reflectors 220 are arranged is further adhered onto the ultrasound transmissive medium 210-4. An ultrasound transmissive medium 210-2 on which multiple reflectors 220 are arranged is then further adhered onto the ultrasound transmissive medium 210-3, and then lastly an ultrasound transmissive medium 210-1 that does not have reflectors 220 is adhered onto the ultrasound transmissive medium 210-2, thus obtaining an ultrasonic measurement sheet 200 in which each reflector group 230 has four reflectors 220.

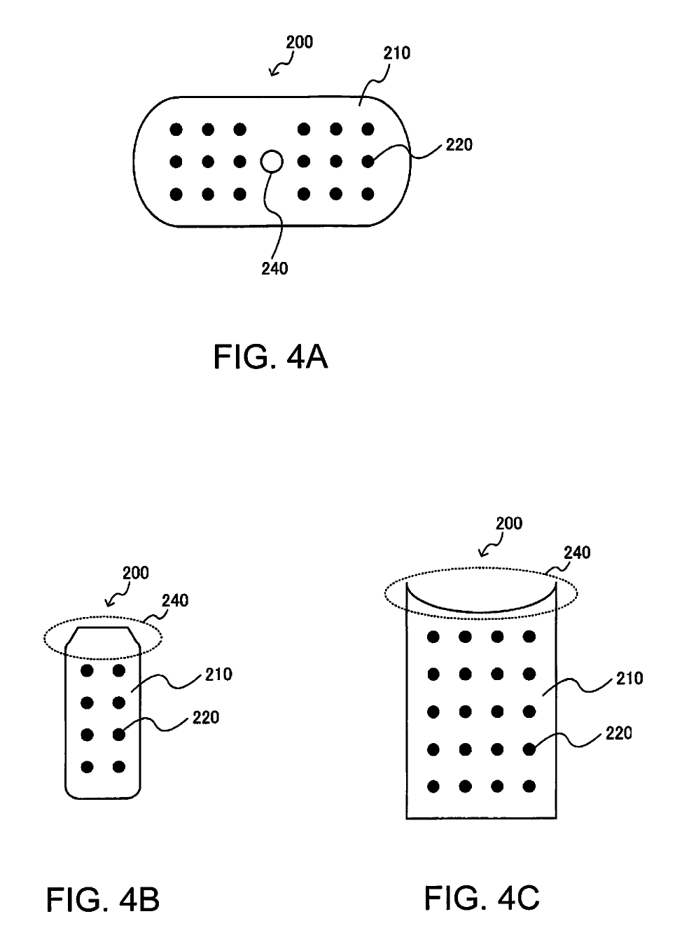

FIGS. 4A to 4C show examples of specific configurations of the ultrasonic measurement sheet 200 of this embodiment. The ultrasonic measurement sheets 200 shown in FIGS. 4A to 4C each include an alignment portion 240. The alignment portion 240 is for alignment with a specific location on the human body, and is provided as a hole, a notch, or the like in a portion of the ultrasonic measurement sheet 200, for example.

FIG. 4A shows an ultrasonic measurement sheet 200 for the abdomen, and a hole for alignment with a person's navel is provided as the alignment portion 240.

FIG. 4B shows an ultrasonic measurement sheet 200 for the arm, and notches for alignment with a person's cubital fossa (the recessed portion formed on the inside of the arm when it is bent) are provided as the alignment portion 240. FIG. 4C shows an ultrasonic measurement sheet 200 for the thigh, and a notch for alignment with a person's knee is provided as the alignment portion 240. Note that the shapes of the ultrasonic measurement sheets 200 shown in FIGS. 4A to 4C are merely examples, and the invention is not limited to the illustrated shapes.

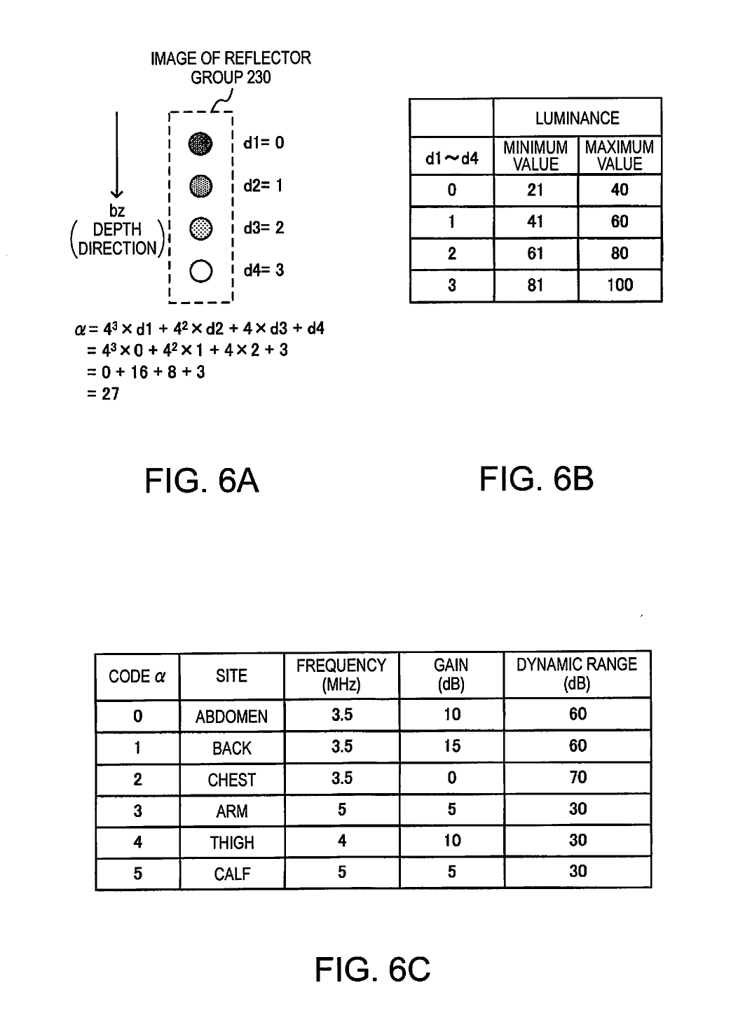

As shown in FIG. 6C, which will be described later, the code information α=0, which corresponds to the abdomen, is recorded in the ultrasonic measurement sheet 200 for the abdomen. Also, the code information α=3, which corresponds to the arm, is recorded in the ultrasonic measurement sheet 200 for the arm. Furthermore, the code information α=4, which corresponds to the thigh, is recorded in the ultrasonic measurement sheet 200 for the thigh.

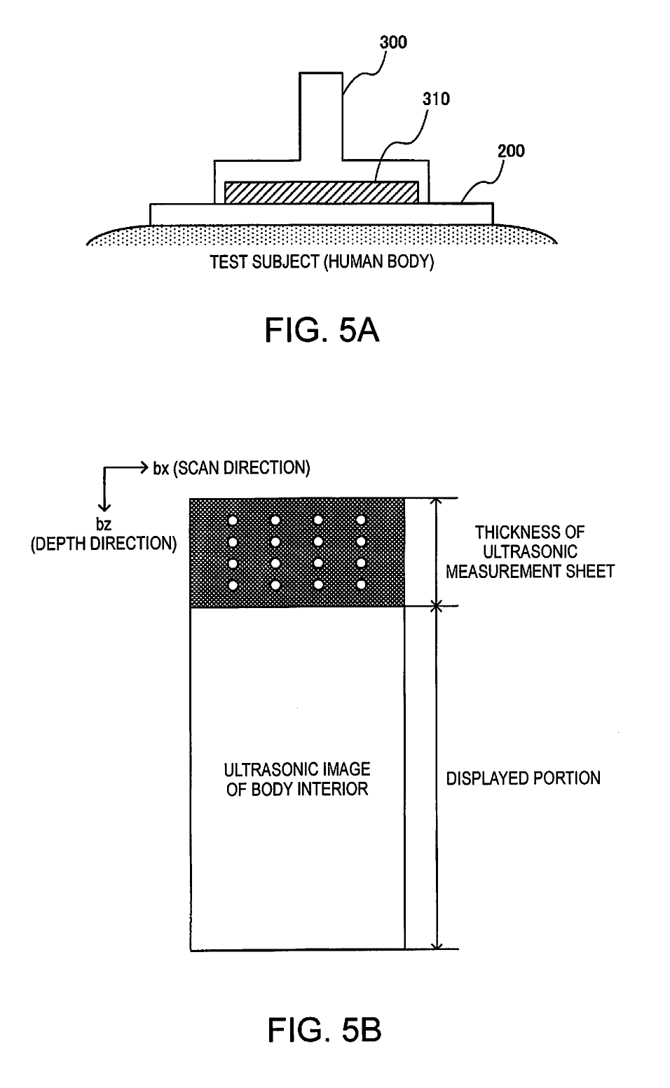

FIG. 5A shows an example of use of the ultrasonic measurement sheet 200 of this embodiment. As shown in FIG. 5A, the ultrasonic measurement sheet 200 is brought into close contact with the test subject (human body), and then the ultrasonic probe 300 is placed on the ultrasonic measurement sheet 200 so as to be in close contact therewith. The ultrasonic probe 300 includes an ultrasonic transducer device 310, and the ultrasonic transducer device 310 emits ultrasonic waves based on an emission signal from the emission unit 110 as well as receives ultrasonic echoes and outputs a reception signal to the reception unit 120. Although not shown, the ultrasonic probe 300 is electrically connected to the ultrasonic diagnosis device 400 via a cable.

FIG. 5B shows an example of an ultrasonic image (B mode image) that is generated by the processing unit 130 based on a reception signal that is based on ultrasonic echoes. In FIG. 5B, the bx direction is the scan direction, and the bz direction is the depth direction.

As shown in FIG. 5B, the region corresponding to the thickness of the ultrasonic measurement sheet 200 has an ultrasonic image of reflectors 220, and the region deeper than that region has an ultrasonic image of the interior of the body (test subject). The processing unit 130 is able to prevent the region corresponding to the thickness of the ultrasonic measurement sheet 200 from being displayed on the display unit 410, and display only the ultrasonic image of the interior of the body on the display unit 410. This configuration enables excluding the region of the image that corresponds to the ultrasonic measurement sheet 200, which is not necessary to the user, from the display.

3. Code Information and Processing for Analysis Thereof.

FIG. 6A shows an example of code information recorded using the reflectance of the reflectors 220. FIG. 6A shows an ultrasonic image (B mode image) of a reflector group 230 made up of four reflectors 220. The reflectance of each reflector 220 is set to any of four levels of reflectances R1, R2, R3, and R4 (R1<R2<R3<R4). The reflectance R4 is the highest, and the reflectance R1 is the lowest. In the B mode image that is obtained, the luminance is higher the higher the reflectance of the subject is, and therefore the image of the reflector 220 with the reflectance R1 has the lowest luminance, and the luminances of the images of the reflectors 220 rise in correspondence with the rising reflectances R2, R3, and R4. The processing unit 130 can analyze the code information recorded by the reflector group 230 through obtaining the luminance of each of the reflectors 220 based on ultrasonic image data.

For each image of a reflector 220, the processing unit 130 determines which of the four luminance levels the luminance (luminance information) of the image corresponds to. A luminance level d is then obtained for each reflector 220 based on the determination results. The luminance level d takes any value among the values “0”, “1”, “2”, and “3”. Next, the processing unit 130 obtains the code information α based on the luminance levels d of the reflectors 220.

FIG. 6B shows an example of a luminance table indicating the correspondence between the luminance level d and image luminance. In FIG. 6B, the reflector 220 luminances are shown as relative values based on the maximum value of “100”. For example, if the luminance of a certain reflector 220 is in the range of “21” to “40”, the luminance level d of that reflector 220 is “0”. Also, if the luminance of a certain reflector 220 is in the range of “61” to “80”, the luminance level d of that reflector 220 is “2”. In this way, the processing unit 130 can obtain the luminance level d for each reflector 220.

In the example shown in FIG. 6A, the luminance levels d1 to d4 of the four reflectors 220 included in the reflector group 230 are d1=0, d2=1, d3=2, and d4=3 in order along the bz direction (depth direction). The processing unit 130 obtains the code information α using the following equation.

α=4³×d1+4²×d2+4×d3+d4 (3)

For example, in the case shown in FIG. 6A, the code information α is α=27. In this way, the code information α=27 is recorded in the reflector group 230 shown in FIG. 6A.

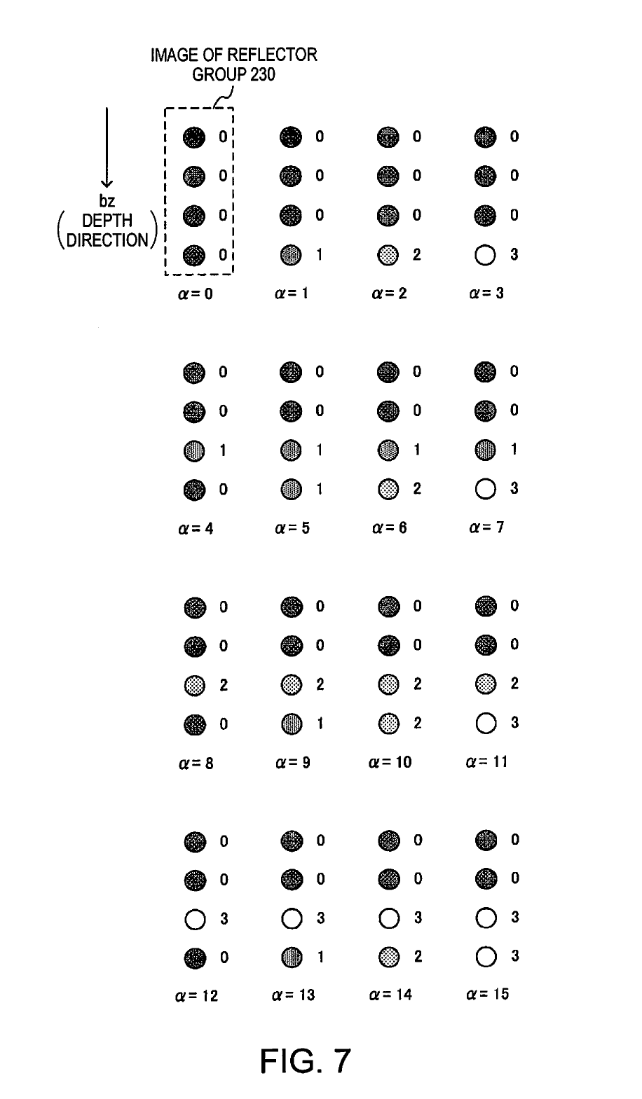

FIG. 7 shows an example of images of reflector groups 230 recording code information α having the values “0” to “15”. Each reflector group 230 includes four reflectors 220 similarly to the case shown in FIG. 6A.

Letting d1, d2, d3, and d4 be the luminance levels d of the four reflectors 220 in order along the bz direction (depth direction): d1=d2=d3=d4=0 in the case of the code information α=0; d1=d2=0, d3=1, and d4=3 in the case of the code information α=7; and d1=d2=0 and d3=d4=2 in the case of the code information α=10, for example. By setting each of the four reflectors 220 to any one of four levels of reflectances, it is possible to record 4⁴=256 types of code information, that is to say α=0 to 255.

In this way, according to the ultrasonic measurement sheet 200 of this embodiment, the code information α can be recorded by setting the reflectances of the reflectors 220 included in a reflector group 230 to predetermined values. The processing unit 130 can then acquire the code information α by performing analysis processing based on the luminance (luminance information) of the ultrasonic image of the reflector group 230.

FIG. 6C shows an example of a reference table in which measurement sites, control parameters, image generation parameters, and the code information α are associated with each other. In FIG. 6C, the ultrasonic frequency is used as the control parameter, and gain and dynamic range are used as the image generation parameters. The processing unit 130 acquires the control parameter and the image generation parameters that correspond to the acquired code information α in accordance with the reference table shown in FIG. 6C, and performs control processing that is appropriate for the measurement site based on the acquired parameters. Specifically, the processing unit 130 controls the emission unit 110 and the reception unit 120 based on the acquired control parameter so as to set the frequency of the ultrasonic waves that are to be emitted and received, and generates ultrasonic image data based on the acquired image generation parameters (gain and dynamic range). The reference table can be stored in advance in a storage unit of the ultrasonic measuring device 100.

For example, in the case of α=0, the measurement site is the abdomen, the frequency is 3.5 MHz, the gain is 10 dB, and the dynamic range is 60 dB. Also, in the case of α=3, the measurement site is the arm, the frequency is 5 MHz, the gain is 5 dB, and the dynamic range is 30 dB. In this way, the code information α is site specification information for specifying the measurement site, and based on this site specification information, the processing unit 130 sets parameters to be used in the measurement of the measurement site, namely a control parameter that is used in control of at least one of the emission unit 110 and the reception unit 120, and an image generation parameter that is used in the generation of ultrasonic image data.

The higher the ultrasonic frequency is, the higher the axial resolution is, and the easier it is to obtain a clear image, but the penetration distance decreases due to an increase in attenuation. Accordingly, it is desirable to use a higher frequency (e.g., 5 MHz) when measuring a shallow portion such as the arm or the calf, and use a lower frequency (e.g., 3.5 MHz) when measuring a deep portion such as the abdomen or the chest.

The gain and the dynamic range are parameters that are used when generating a B mode image, which will be described later, and are for setting the signal level range of the reception signal that is to be displayed using luminance. The values of the gain and the dynamic range that are desirable differ depending on the measurement site.

According to the ultrasonic measuring device 100 and the ultrasonic measurement sheet 200 of this embodiment, the processing unit 130 can automatically identify a measurement site using the code information recorded by a reflector group 230, and set a frequency, a gain, a dynamic range, and the like that are appropriate for the identified measurement site.

Note that although the processing unit 130 acquires the control parameter and the image generation parameters using a reference table in the above-described example, a configuration is possible in which a reference table is not used, and the parameter values are directly recorded as values making up the code information a.

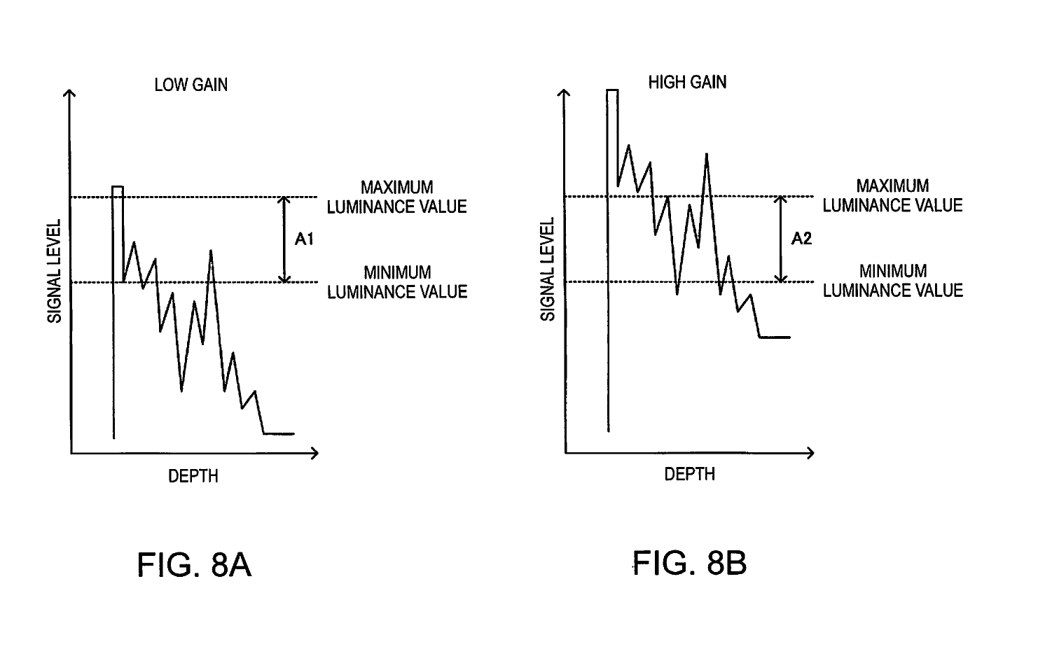

FIGS. 8A and 8B are diagrams for describing gain, which is one of the image generation parameters. In FIGS. 8A and 8B, the signal level of the reception signal obtained by logarithmic transformation after wave detection is plotted against depth. As shown in FIG. 8A, the image data is generated based on a range A1, that is to say the portion of the signal between the maximum and minimum luminance values that are to be displayed in a B mode image. However, if the gain is higher than that in FIG. 8A, as in FIG. 8B, image data is generated based on the signal in a range A2. Note that portions of the signal that exceed the maximum luminance value are treated as having the maximum luminance value, and portions of the signal that are lower than the minimum luminance value are treated as having the minimum luminance value.

In this way, as the gain is increased, an ultrasonic image corresponding to a weaker reception signal (i.e., ultrasonic echoes with lower amplitudes) can be generated. On the other hand, as the gain is reduced, an ultrasonic image corresponding to a stronger reception signal (i.e., ultrasonic echoes with higher amplitudes) can be generated.

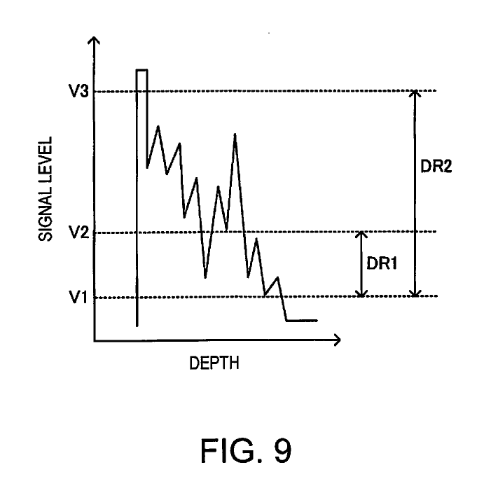

FIG. 9 is a diagram for describing dynamic range, which is one of the image generation parameters. In FIG. 9, the signal level of the reception signal obtained by logarithmic transformation after wave detection is plotted against depth. In the case of a first dynamic range DR1, image data is generated based on the portion of the signal in which the signal level is in the range V1 to V2. Also, in the case of a second dynamic range DR2 that is higher than the first dynamic range DR1, image data is generated based on a portion of the signal in which the signal level is in the range V1 to V3 (V3>V2).

In this way, raising the dynamic range enables displaying a wide signal range, from weak signals to strong signals, but on the other hand, the image will have low contrast since minute differences in the signal level will not be represented. On the other hand, lowering the dynamic range reduces the range of signal levels that can be displayed, but enables obtaining an image with high contrast for a specific subject.

The gain and the dynamic range that are desirable differ depending on the measurement site, and it is difficult for a user lacking expertise to set a gain and a dynamic range that are appropriate for the measurement site. According to the ultrasonic measuring device 100 of this embodiment, it is possible to analyze code information recorded by a reflector group 230 and automatically set a gain, a dynamic range, and the like that are appropriate for the measurement site.

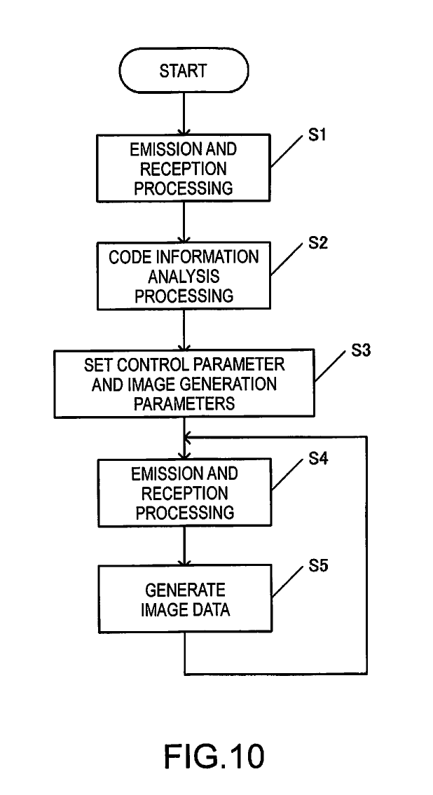

FIG. 10 shows an example of a flowchart of measurement processing performed by the ultrasonic measuring device 100 of this embodiment. The processing flow shown in FIG. 10 is executed based on control processing performed by the processing unit 130.

In a first period, the processing unit 130 performs processing for analyzing code information, and based on the code information, sets a control parameter that is used in the control of at least one of the emission unit 110 and the reception unit 120, and image generation parameters for the generation of ultrasonic image data. Then, in a second period that is after the first period, the processing unit 130 controls at least one of the emission unit 110 and the reception unit 120 based on the control parameter that was set, and generates ultrasonic image data based on the image generation parameters that were set. In the flowchart shown in FIG. 10, steps S1 to S3 are executed in the first period, and steps S4 and S5 are executed in the second period.

The first period is the period in which the processing unit 130 sets the parameters that are to be used in ultrasonic measurement, and the second period is the period in which the processing unit 130 performs processing for controlling ultrasonic measurement based on the parameters that were set.

Firstly, emission and reception processing is performed (step S1). This emission and reception processing is performed in order to acquire ultrasonic image data for the analysis of the code information recorded in the ultrasonic measurement sheet 200. In this emission and reception processing, the control parameter and the image generation parameters are set such that differences between the reflectances of the reflectors 220 in the ultrasonic measurement sheet 200 are shown clearly.

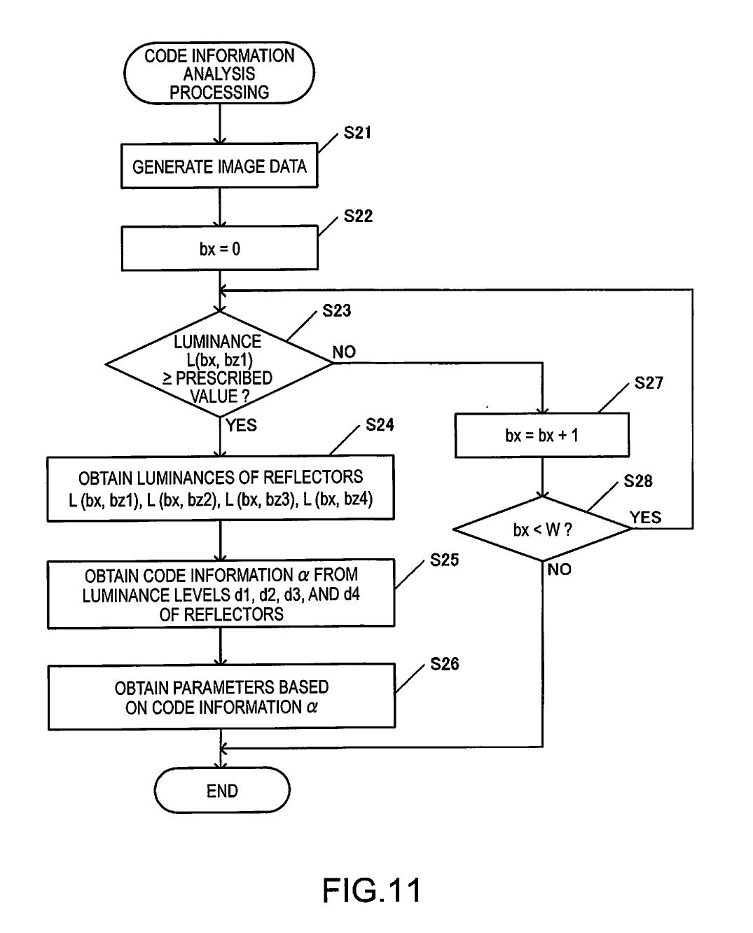

Next, processing for analyzing code information is performed (step S2). This analysis processing will be described in detail later with reference to FIG. 11.

Next, the control parameter and the image generation parameters are set based on the acquired code information α (step S3). Specifically, the frequency, the gain, the dynamic range, and the like that correspond to the code information α based on the reference table shown in FIG. 6C, for example, are set.

Subsequently, emission and reception processing is performed using the parameters that were set (step S4), and image data is generated (step S5). The procedure then returns to step S4, and emission and reception processing is performed for the next frame image.

FIG. 11 shows an example of a flowchart of code information analysis processing performed by the ultrasonic measuring device 100 of this embodiment. The processing flow shown in FIG. 11 is executed by the processing unit 130.

Firstly, the processing unit 130 generates ultrasonic image data (B mode image data) based on the reception signal (step S21). This image data corresponds to the ultrasonic image shown in FIG. 5B, for example. As shown in FIG. 5B, the scan direction in the B mode image is the bx direction, and the depth direction is the bz direction.

Next, the processing unit 130 sets a scan direction coordinate value bx to an initial value (e.g., bx=0) (step S22). Specifically, this scan direction coordinate value bx can be expressed in units of image pixels. For example, bx=n (n being an integer greater than or equal to 0) corresponds to the (n+1)-th pixel along the scan direction from the origin of the image.

Next, the processing unit 130 obtains, from the image data, a luminance L(bx,bz1) of the pixel that corresponds to the scan direction coordinate value bx and a depth direction coordinate value bz1. The processing unit 130 then determines whether or not the luminance L(bx,bz1) is greater than or equal to a prescribed value (step S23). Here, bz1 is the depth direction coordinate value bz of the pixels that correspond to the images of the reflectors 220 that are at the most shallow position. Similarly to bx, the depth direction coordinate value bz can also be expressed in units of pixels. If the luminance L(bx,bz1) is greater than or equal to the prescribed value, the procedure moves to step S24. Here, the prescribed value is the minimum luminance value of a reflector 220 image for example, and in the example shown in FIG. 6B, the minimum luminance value (relative value) is specifically “21”.

In step S24, the processing unit 130 obtains luminances L(bx,bz1), L(bx,bz2), L(bx,bz3), and L(bx,bz4) of four reflectors 220 having the same scan direction coordinate value bx and different depth direction coordinate values bz. Here, the relationship bz1<bz2<bz3<bz4 is satisfied.

Subsequently, the processing unit 130 obtains the luminance levels d1, d2, d3, and d4 that correspond to the luminances L(bx,bz1), L(bx,bz2), L(bx,bz3), and L(bx,bz4) of the four reflectors 220, and furthermore obtains the code information α from the luminance levels d1, d2, d3, and d4 (step S25). The processing unit 130 then obtains the control parameter and the image generation parameters based on the code information α (step S26). Specifically, the processing unit 130 can obtain the luminance levels d1, d2, d3, and d4 using the luminance table shown in FIG. 6B for example, and can obtain the control parameter and image generation parameters that are appropriate for the measurement site using the reference table shown in FIG. 6C for example.

On the other hand, if the luminance L(bx,bz1) is less than the prescribed value, the pixel does not correspond to a reflector 220 image, and therefore the processing unit 130 increments the scan direction coordinate value bx, that is to say bx=bx+1 (step S27). The processing unit 130 then determines whether or not the incremented value of bx is lower than a scan width W (step S28). The scan width W is a value that corresponds to the number of pixels along the scan direction in the ultrasonic image, for example. If the incremented value of bx is lower than the scan width W, the procedure returns to step S23, and the processing unit 130 determines whether or not the luminance L(bx,bz1) is greater than or equal to the prescribed value. Here, if the luminance is again less than the prescribed value, the processing unit 130 again increments the value of bx. In this way, the processing unit 130 increments the value of bx until the luminance L(bx,bz1) is greater than or equal to the prescribed value, and the processing target pixel moves along the scan direction.

If it is determined that the value of bx incremented in this way is greater than or equal to the scan width W, this procedure ends without code information analysis processing being performed since the processing unit 130 was not able to find a reflector 220 image.

As described above, according to the ultrasonic measuring device 100 and the ultrasonic measurement sheet 200 of this embodiment, the processing unit 130 can analyze code information recorded by reflectors 220, identify the measurement site based on the analyzed code information, and set a control parameter (e.g., the frequency), image generation parameters (e.g., the gain and the dynamic range), and the like that are appropriate for the measurement site.

4. Ultrasonic Diagnosis Device

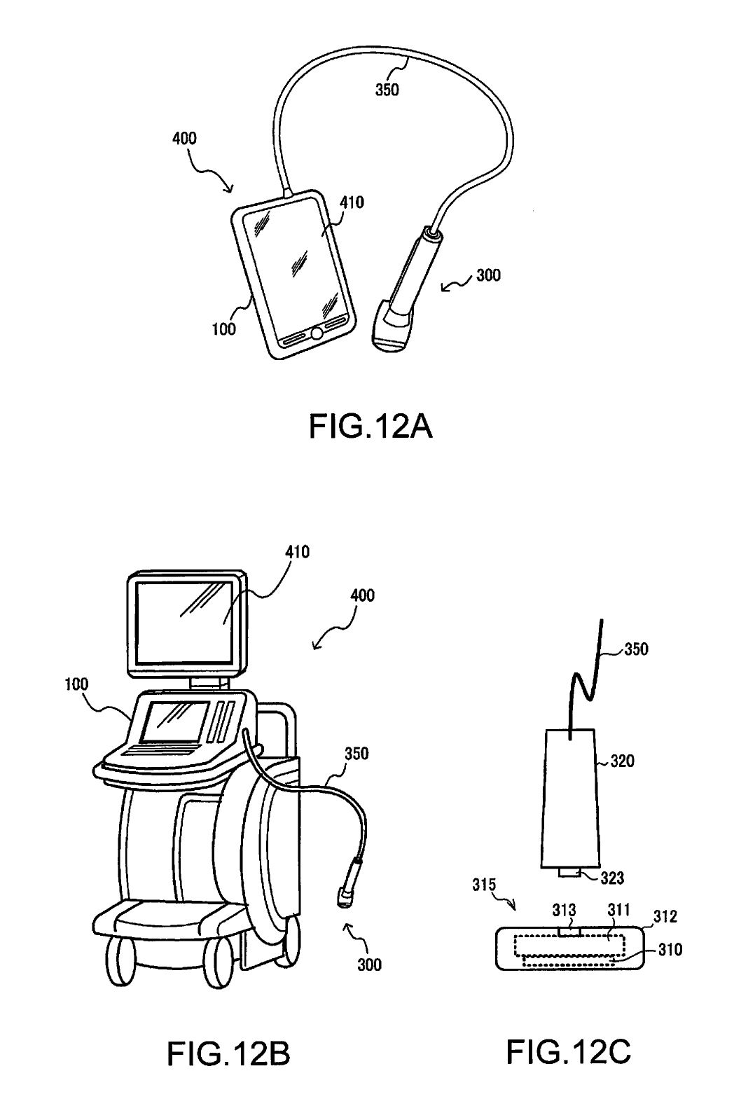

FIGS. 12A and 12B show examples of specific configurations of the ultrasonic diagnosis device 400 of this embodiment. FIG. 12A shows a portable ultrasonic diagnosis device 400, and FIG. 12B shows a stationary ultrasonic diagnosis device 400.

The portable and stationary ultrasonic diagnosis devices 400 both include the ultrasonic measuring device 100, the ultrasonic probe 300, a cable 350, and the display unit 410. The ultrasonic probe 300 includes the ultrasonic transducer device 310 and is connected to the ultrasonic measuring device 100 via the cable 350. The display unit 410 displays display image data.

At least a portion of the emission unit 110, the reception unit 120, and the processing unit 130 of the ultrasonic measuring device 100 can be provided in the ultrasonic probe 300.

FIG. 12C shows an example of the specific configuration of the ultrasonic probe 300 of this embodiment. The ultrasonic probe 300 includes a probe head 315 and a probe body 320, and as shown in FIG. 12C, the probe head 315 is detachable from the probe body 320.

The probe head 315 includes the ultrasonic transducer device 310, a probe base 311, a probe housing 312, and a probe head-side connector 313.

The probe body 320 includes a probe body-side connector 323. The probe body-side connector 323 is connected to the probe head-side connector 313. The probe body 320 is connected to the ultrasonic measuring device 100 via the cable 350. Note that at least a portion of the emission unit 110 and the reception unit 120 of the ultrasonic measuring device 100 can be provided in the probe body 320.

Note that although various embodiments have been explained in detail above, a person skilled in the art will readily appreciate that it is possible to implement numerous variations and modifications that do not depart substantially from the novel aspects and effect of the invention. Accordingly, all such variations and modifications are also to be included within the scope of the invention. For example, terms that are used within the description or drawings at least once together with broader terms or alternative synonymous terms can be replaced by those other terms at other locations as well within the description or drawings. Also the configuration and operation of the ultrasonic measuring device, the ultrasonic diagnosis device, and the ultrasonic measurement sheet are not limited to those described in the embodiments, and various modifications are possible.

The entire disclosure of Japanese Patent Application No. 2013-004302, filed Jan. 15, 2013 is expressly incorporated by reference herein.

What is claimed is:

1. An ultrasonic measuring device comprising:
an emission unit that performs ultrasound emission processing;
a reception unit that performs ultrasonic echo reception processing; and
a processing unit that performs ultrasonic measurement control processing,
wherein the emission unit performs processing for emitting ultrasound toward a subject via an ultrasonic measurement sheet,
the reception unit performs processing for receiving an ultrasonic echo from the ultrasonic measurement sheet, and outputs a reception signal to the processing unit, and
the processing unit performs processing for analyzing ultrasonic measurement code information recorded in the ultrasonic measurement sheet based on the reception signal from the reception unit.

2. The ultrasonic measuring device according to claim 1,
wherein the processing unit sets a control parameter that is used in control of at least one of the emission unit and the reception unit based on the code information, and controls at least one of the emission unit and the reception unit based on the control parameter.

3. The ultrasonic measuring device according to claim 1,
wherein the processing unit sets an image generation parameter for generation of ultrasonic image data based on the code information, and generates the ultrasonic image data based on the image generation parameter.

4. The ultrasonic measuring device according to claim 3,
wherein the image generation parameter is a parameter for setting at least one of gain and dynamic range.

5. The ultrasonic measuring device according to claim 1,
wherein in a first period, the processing unit performs the code information analysis processing and, based on the code information, sets a control parameter that is used in control of at least one of the emission unit and the reception unit and an image generation parameter for generation of ultrasonic image data, and in a second period that is after the first period, the processing unit controls at least one of the emission unit and the reception unit based on the control parameter, and generates the ultrasonic image data based on the image generation parameter.

6. The ultrasonic measuring device according to claim 5,
wherein the first period is a period in which the processing unit sets a parameter to be used in ultrasonic measurement, and
the second period is a period in which the processing unit performs ultrasonic measurement control processing based on the parameter that was set.

7. The ultrasonic measuring device according to claim 1,
wherein the code information is site specification information for specifying a measurement site.

8. The ultrasonic measuring device according to claim 7,
wherein based on the site specification information, the processing unit sets a control parameter that is used in control of at least one of the emission unit and the reception unit and an image generation parameter for generation of ultrasonic image data, the control parameter and the image generation parameter being parameters that are used in measurement of the measurement site.

9. The ultrasonic measuring device according to claim 1,
wherein the processing unit acquires at least one of characteristic information and manufacturing information of the ultrasonic measurement sheet based on the code information.

10. The ultrasonic measuring device according to claim 1,
wherein the ultrasonic measurement sheet has an ultrasound transmissive medium and a plurality of reflectors embedded in the ultrasound transmissive medium,
the code information is recorded using at least one of the reflectance, the number, the shape, and the size of the plurality of reflectors,
the reception unit performs processing for receiving an ultrasonic echo from the plurality of reflectors, and outputs a reception signal to the processing unit, and
the processing unit performs processing for analyzing the code information based on the reception signal from the reception unit.

11. The ultrasonic measuring device according to claim 10,
wherein the ultrasonic measurement sheet has a plurality of reflector groups aligned in the ultrasound transmissive medium as the plurality of reflectors,
each reflector group among the plurality of reflector groups has 1st to p-th (p being an integer greater than or equal to 2) reflectors that are aligned along a depth direction of the ultrasonic measurement sheet, and
the processing unit performs processing for analyzing the code information recorded by the 1st to p-th reflectors.

12. The ultrasonic measuring device according to claim 11,
wherein the same code information is recorded by each reflector group among the plurality of reflector groups, and
the processing unit performs processing for analyzing the code information recorded by at least one reflector group among the plurality of reflector groups.

13. An ultrasonic diagnosis device comprising:
the ultrasonic measuring device according to claim 1; and
a display unit that displays display image data.

14. An ultrasonic measurement sheet comprising:
an ultrasound transmissive medium; and
a plurality of reflectors embedded in the ultrasound transmissive medium,
wherein code information that is to be analyzed in analysis processing performed by an ultrasonic measuring device is recorded using at least one of the reflectance, the number, the shape, and the size of the plurality of reflectors.

15. The ultrasonic measurement sheet according to claim 14,
wherein the ultrasonic measurement sheet includes a plurality of reflector groups aligned in the ultrasound transmissive medium as the plurality of reflectors,
each reflector group among the plurality of reflector groups has 1st to p-th (p being an integer greater than or equal to 2) reflectors that are aligned along a depth direction of the ultrasonic measurement sheet, and
the code information is recorded by the 1st to p-th reflectors.

16. A measuring method comprising the steps of:
transmitting ultrasound toward a subject via an ultrasonic measurement sheet;
receiving an ultrasonic echo from the ultrasonic measurement sheet; and
performing processing for analyzing ultrasonic measurement code information recorded in the ultrasonic measurement sheet based on a reception signal.
